(19) United States
(12) Patent Application Publication
(10) Pub. No.: US 2013/0131422 A1
(43) Pub. Date: May 23, 2013

(54) SYSTEMS AND METHODS FOR COMMUNICATING DOSE CALIBRATION INFORMATION

(71) Applicants: Kenneth Joseph Vosniak, Wauwatosa, WI (US); Alexander Ganin, Whitefish Bay, WI (US); Joan Hanson, Cambridge, WI (US); Paul Kinahan, Seattle, WA (US); Andrew Strickland, Bellevue, WA (US)

(72) Inventors: Kenneth Joseph Vosniak, Wauwatosa, WI (US); Alexander Ganin, Whitefish Bay, WI (US); Joan Hanson, Cambridge, WI (US); Paul Kinahan, Seattle, WA (US); Andrew Strickland, Bellevue, WA (US)

(73) Assignee: General Electric Company, Schenectady, NY (US)

(21) Appl. No.: 13/304,263

(22) Filed: Nov. 23, 2011

(51) Int. Cl. A61N 5/00; G01T 1/164

(52) U.S. Cl. 600/1; 250/362; 250/363.03

(57) ABSTRACT

Systems and methods for communicating dose calibration information are provided. One method includes determining dose calibration information of a radiopharmaceutical at a dose calibrator. The method also includes automatically storing the dose calibration information in a memory. The method further includes communicating the stored dose calibration information to a host system.

### BACKGROUND OF THE INVENTION

The subject matter disclosed herein relates generally to dose calibrators, and more particularly to communicating dose calibration information to medical imaging scanners, which may be used when reconstructing or forming images.

Radionuclides used in positron emission tomography (PET) or single photon emission computed tomography (SPECT) scanning are typically isotopes with short half-lives such as carbon-11 (approximately 20 min), nitrogen-13 (approximately 10 min), oxygen-15 (approximately 2 min), and fluorine-18 (approximately 110 min). These radionuclides are incorporated into compounds normally used by the body such as glucose (or glucose analogues), water, or ammonia, or into molecules that bind to receptors or other sites of drug action. Such labeled compounds are known as radiotracers and/or radiopharmaceuticals.

In a conventional PET imaging or SPECT imaging control system, an individual dose of a premeasured radiopharmaceutical is administered to a patient. The individual premeasured radiopharmaceutical is prepared by a radiotracer supplier (commonly called a radiopharmacy). The radiotracer is delivered to a medical facility that administers the individual premeasured radiopharmaceutical in accordance with a prescription from a physician. Alternatively, the individual dose may be drawn from a larger batch of a radiotracer on site, also in accordance with a prescription from a physician.

Additionally, in a clinical work flow, the radiopharmaceutical dose activity is measured using a dose calibrator. The measurements made using the dose calibrator may be used to calibrate the scanner to gather data representing radioactivity of the radiopharmaceutical. Additionally, the dose calibration information may be used for post-processing to calculate different values or reconstruct images. For example, standardized uptake values (SUVs), which are a measure of the relative amount of tracer uptake in the patient, may be calculated to assess tumor malignancy or to determine a patient's response to therapy.

In conventional systems it is necessary to transfer the radiopharmaceutical activity measurements data to a host system of the scanner (e.g., Positron Emission Tomography (PET) scanner or Single Positron Emission Computed Tomography (SPECT) scanner) prior to a medical procedure and after the medical procedure. In these systems, the data is typically recorded on paper, carried to the host system and manually re-entered. As a result, in conventional methods using handwritten data or notes, the is a possibility of potential errors in recording and/or re-entering the data, which can cause errors in subsequent post-processing, such as the determination of radiopharmaceutical uptake in tumors and also in the conversion of the image data to SUVs.

### SUMMARY OF THE INVENTION

In accordance with one embodiment, a method for communicating dose calibration information is provided. The method includes determining dose calibration information of a radiopharmaceutical at a dose calibrator, automatically storing the dose calibration information in a memory and communicating the stored dose calibration information to a host system.

In accordance with another embodiment, a system for communicating radiopharmaceutical activity information is provided that includes a dose calibrator to determine dose calibration information of a radiopharmaceutical. The system also includes a storage device for storing the dose calibration information, wherein the dose calibrator is configured to automatically store the dose calibration information within the storage device. The system further includes a host system communicatively coupled to the dose calibrator.

### BRIEF DESCRIPTION OF THE DRAWINGS

The drawings, in which like numerals represent similar parts, illustrate generally, by way of example, but not by way of limitation, various embodiments discussed in the present document.

FIG. 1 is a block diagram illustrating a radiopharmaceutical communication system in accordance with an embodiment.

FIG. 2 is a diagram illustrating network communication between a dose calibrator and a Positron Emission Tomography (PET) scanner in accordance with an embodiment.

FIG. 3 is a diagram illustrating pre-patient data processing for calculating a standardized uptake value (SUV) in accordance with an embodiment.

FIG. 4 are SUV images illustrating reconstruction with and without calibration error.

FIG. 5 is a flowchart of a method for communicating dose calibration information in accordance with an embodiment.

FIG. 6 is a perspective view of a multi-modality imaging system formed in accordance with various embodiments.

FIG. 7 is a schematic block diagram of a portion of the exemplary imaging system shown in FIG. 6 in accordance with various embodiments.

FIG. 8 is a schematic block diagram of another portion of the exemplary imaging system shown in FIG. 6 in accordance with various embodiments.

### DETAILED DESCRIPTION OF THE INVENTION

The foregoing summary, as well as the following detailed description of certain embodiments of the subject matter set forth herein, will be better understood when read in conjunction with the appended drawings. As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

In the following detailed description, reference is made to the accompanying drawings which form a part hereof, and in which are shown by way of illustration specific embodiments in which the subject matter disclosed herein may be practiced. These embodiments, which are also referred to herein as “examples,” are described in sufficient detail to enable those skilled in the art to practice the subject matter disclosed herein. It is to be understood that the embodiments may be combined or that other embodiments may be utilized, and that structural, logical, and electrical variations may be made without departing from the scope of the subject matter disclosed herein. The following detailed description is, therefore, not to be taken in a limiting sense, and the scope of the subject matter disclosed herein is defined by the appended claims and their equivalents.

In the description that follows, like numerals or reference designators will be used to refer to like parts or elements throughout. In this document, the terms “a” or “an” are used to include one or more than one and the term “or” is used to refer to a nonexclusive or, unless otherwise indicated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Additionally, as used herein, the terms “command” and “signal” may be used interchangeably. Also, the terms radioisotope, radiotracer, radionuclide and radiopharmaceutical may be used interchangeably.

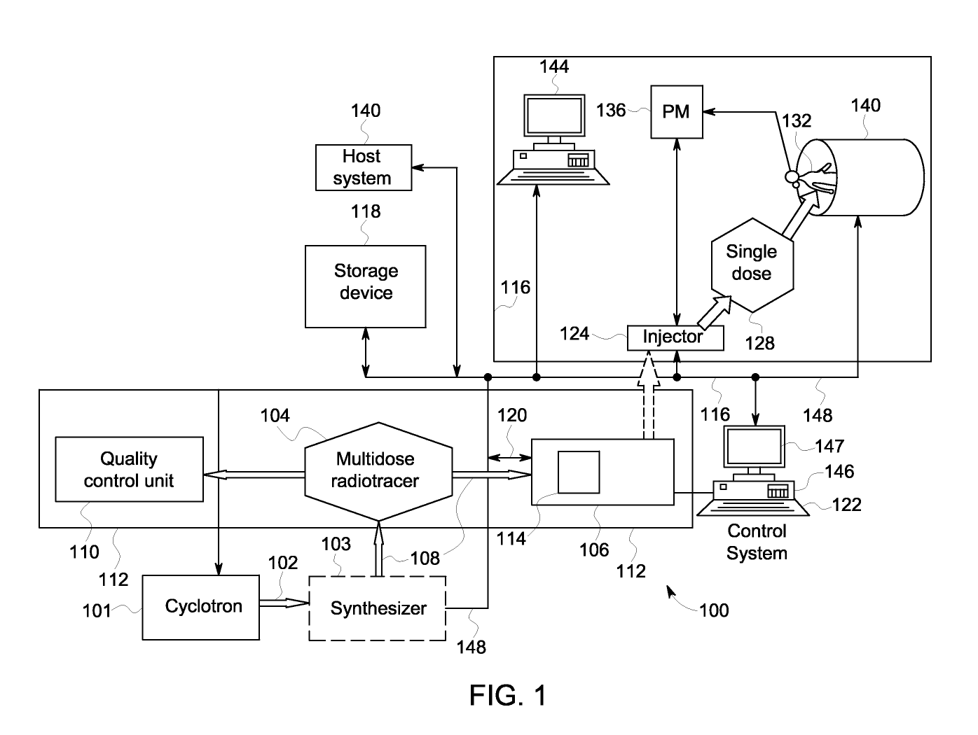

FIG. 1 is a block diagram of a system 100 for communicating radiopharmaceutical activity information, such as dose calibration information, in accordance with an embodiment. In one embodiment, the system 100 may be an integrated system for the production, quality control and distribution of medical radiopharmaceuticals in an imaging system. For example, the imaging system may be a Positron Emission Tomography (PET) imaging system, or a Single Positron Emission Computed Tomography (SPECT) imaging system, among others.

In one embodiment, a radioisotope 102 is produced by a radioisotope generator. The radioisotope 102 is chemically bonded to a biological compound in a chemical synthesizer 103, producing a radiotracer/radiopharmaceutical 104, illustrated as a multidose radiotracer. The radioisotope 102 or the radiotracer 104 is transferred using any suitable method to a dispensing station 106 for storage and administration to patient. The system 100 also includes a quality control unit 110 that monitors the amount of radioactivity and other measures of quality and quantity of the radioisotope 102 that is stored in the dispensing station 106. The quality control unit 110 allows the radionucleic and chemical purity, which is the quality of the radioisotope 102 in terms of the amount of radioactivity of the desired isotope, and chemical purity of the radiotracer, to be verified. The quality control monitoring, analysis and verification may be performed at defined time intervals, for particular production batches or for one or more representative samples of a bulk produced radiotracer. The time intervals and batches can be defined/determined and modified by an operator.

In an exemplary embodiment, the quality control unit 110 includes a high performance liquid chromatography (HPLC) device and/or a Sodium Iodine (NaI) detector. The quality control unit 110 also includes a filter for the radioisotope 102 that is stored in the dispensing station 106.

The dispensing station 106 further includes a dose calibrator 114. The dose calibrator 114 may include an ionization chamber within which a radioisotope 102, a radiotracer 104, or any radiopharmaceutical may be placed to measure the amount of radioactivity of the radiopharmaceutical. For example, the radioactivity of the radiopharmaceutical may be measured before administering the radiopharmaceutical to a subject, for example, a patient 132 and/or after administering the radiopharmaceutical to the patient 132. Optionally, the dispensing station 106 may further include a plurality of dose calibrators 114, such that each of the plurality of dose calibrators 114 may provide dose calibration information. In another embodiment, the dose calibrator 114 may be a stand alone component of the system 100 separate from the dispensing station 106.

Additionally, the dose calibrator 114 may automatically store dose calibration information in a memory, such as a storage device 118 within or in connection with the dose calibrator 114. For example, the storage device 118 may be directly coupled to the dose calibrator 114, form an integral part of the dose calibrator 114 or be a portable storage device. Optionally, the storage device 118 may be coupled to the dose calibrator 114 via a network 120. The network 120 may be any suitable data communication network, which may be a wired network or a wireless network. Thus, various embodiments may automatically extract measurement data, store that data in some location (e.g., a remote or integrated storage device) and communicate the data to a host system. The data may be communicated directly and used, for example, to check that correct measurements are made.

In an exemplary embodiment, the dose calibration information, stored on the storage device 118 may be transferred via the network 120 to a host system 140. For example, the host system 140 may be a data post-processing system or workstation, which may form part of, for example, a PET imaging system or a SPECT imaging system (or optionally a multi-modality imaging system, for example, a PET/CT system).

In one embodiment, the host system 140 may include an image reconstruction system. For example, the image reconstruction system may be a software based data reconstruction system. In another embodiment, the host system 140 may be an information system, such as an online data repository. It should be noted that the dose calibration information may be exchanged among different systems, such as a number of clinical systems, for example, imaging products (RIS/PACS—Radiology Information System/Picture Archiving and Communication System), Hospital Information Systems (HIS), Electronic Medical Records (EMR) systems, Laboratories, Pharmacies and other devices involved in diagnostics or patient monitoring.

The host system 140 may be in a close proximity to (e.g., in the same room as) the dose calibrator 114. Optionally, the host system 140 may be at a remote location. The dose calibrator 114 may communicate the stored dose calibration information to the host system 140 over any suitable communication link, such as a Universal Serial Bus (USB), a recommended standard 232 (RS-232) interface, an Ethernet and the like. However, data may be communicated in any suitable manner, such as via the “cloud” (also referred to as cloud computing or a cloud computing network) using a secure connection. Different communication means may be provided, for example, if another communication method is not available. The dose calibrator 114 may use any combination of data communication methods or links, such as USB, RS-232, Ethernet and the like to communicate with the host system 140. Optionally, the dose calibrator 114 may communicate with the host system using a wireless network.

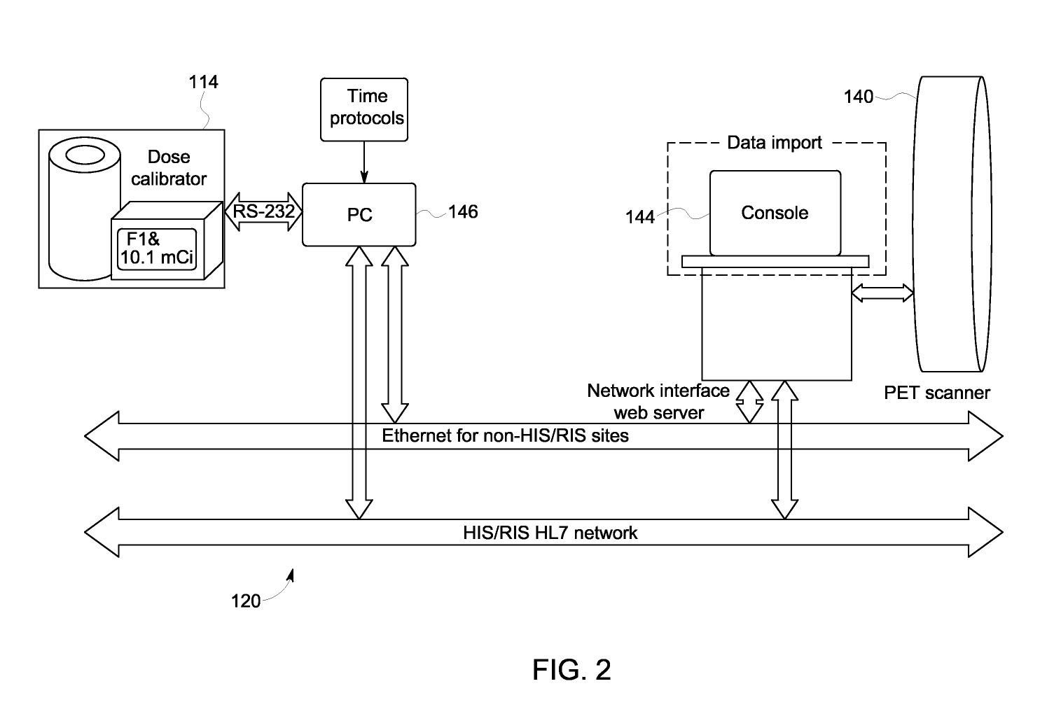

FIG. 2 illustrates an exemplary embodiment of a network communication 120 between the dose calibrator 114 and a PET scanner 140. The dose calibrator 114 may communicate the stored dose calibration information in different formats, for example, encoded in a DICOM file format. For example, the dose calibration information may be encoded in DICOM files as a private tag or a work-list or any combination thereof, as well as other known methods. The dose calibrator 114 may also communicate the stored dose calibration information encoded in a flat file format, printable barcode format, or any combination thereof. In one exemplary embodiment, a camera may be used such that the camera captures an image of dose calibrator readout information. The readout information may be a digital display. The captured image of read-out information may be converted into an alphanumerical string, such that the alphanumerical string may be communicated and stored on the storage device 118 (shown in FIG. 1).

Referring again to FIG. 1, the system 100 further includes a user interface 122 for interactively communicating with a user. In one embodiment, the user interface 122 (that may also include an integrated display) is provided to receive commands from a user and to instruct a processing unit 146 to display on a display 147 a reconstructed image data on an integrated display and/or send the acquired raw data to the storage device 118, in accordance with the commands from the user. The user interface 122 may also be used to input patient information at a location where the dose calibrator 114 is located. Additionally, the user interface 122 may allow a user to link or identify information, such as patient information and dose calibration information (e.g., date, time and dose measurement information).

In one embodiment, a typewriter-like keyboard of buttons is included in user interface 122, as well as one or more soft keys that may be assigned functions in accordance with the mode of operation of the system 100. A portion of the display 147 may be devoted to labels for soft keys. The user interface 122 may also have additional keys and/or controls for special purpose functions, which may include, but are not limited to “patient information display,” “query for patient information,” “a scan patient command,” “print,” and “store.”

The system 100 further includes an injector system 124. The injector system 124 may extract a single dose 128 of a radiopharmaceutical and inject or deliver the dose into the patient 132. It should be noted that instead of the patient 132, the subject may be an animal or a phantom for research purposes. The system 100 also may allow a multi-dose portion of the radiotracer 104 to be dispensed as the single dose 128.

It should be noted that in one embodiment the injector system 124 is an automated injector. However, in other embodiments, a technologist manually injects the radiopharmaceutical into the patient with a syringe. In this manual injection case the radiopharmaceutical activity is calibrated at a time-stamped moment and the user enters the time of the injection, for example, using a graphical user interface (GUI) or the time-stamping may be automatically provided.

Thus, by practicing at least one embodiment, errors associated with recording radiopharmaceutical activity measurements and re-entry of the recorded data for further processing may be reduced. Additionally, an automatic process may be provided that is more reliable, and has less human interaction, as well as reducing the number of tasks performed by an imaging technologist.

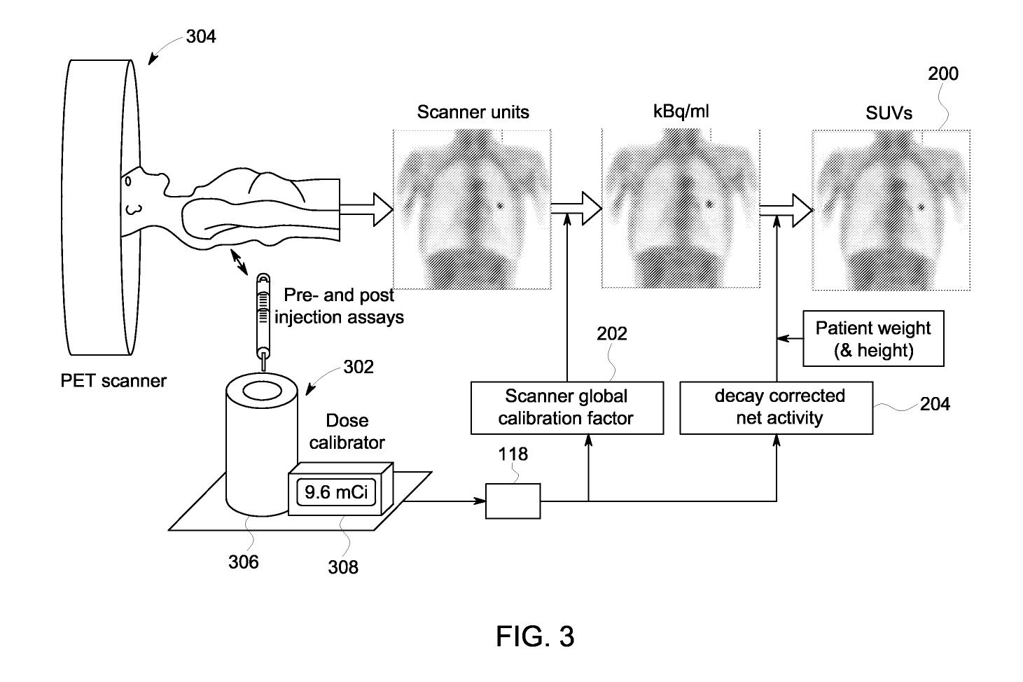

FIG. 3 illustrates pre-patient data processing for SUVs. As illustrated, a plurality of data steps are performed per patient 132, illustrated as fourteen data steps, associated with the radiopharmaceutical dose measurements for producing SUV images 200 during each patient imaging procedure. For example, a plurality of steps may be performed in connection with radiopharmaceutical dose measurements 302 during a calibration of an imaging system 304. For example, a first set of steps include measuring radiopharmaceutical activity at 306, recording radiopharmaceutical activity at 308, recording a time the radiopharmaceutical activity is measured at 308, recording an injection time at 308, measuring residual radiopharmaceutical activity at 306, recording residual radiopharmaceutical activity at 308, and recording a residual measurement time at 308. Using the measured and recorded data, calibration factors 202 and decay corrected net activity information 204 may be determined using suitable methods. In various embodiments, the measured and recorded data is performed electronically and automatically stored and communicated, such as to the host system 140 (shown in FIG. 1).

Returning to FIG. 1, in one embodiment, a physiologic monitoring device (PM) 136 also may be operably coupled to the injector system 124 and to the patient 132, respectively. The PM 136 monitors, for example, a number of measures of the health of the subject, such as blood pressure and heart activity as represented by an electrocardiogram (EKG). The PM 136 may detect abnormalities in the measures of the health of the patient 132 and provide notice of the abnormalities to one or more control systems, as well to clinical staff.

In operation, the patient 132 is placed inside a scanner 140 after or during injection of the radiopharmaceutical 128 to detect the radioactivity of the injected radiopharmaceutical 128 in the patient 132. In one embodiment, a computer 144 with a GUI that is located at the imaging system 116 may be provided to allow a technician to manage, control, and oversee the entire imaging process, including activities of the injector system 124, such as dispensing and injection of the individual dose of radiopharmaceutical 128 into patient 132 and scanning the patient 132 using appropriate clinical protocol.

In one embodiment, the computer 144 receives notification from the PM 136 of abnormalities in the measurements of the health of the patient 132, and instructs the injector system 124 respectively to halt infusion or take other appropriate action.

The computer 144 also may instruct the scanner 140 to initiate a scanning operation at an appropriate time after infusion by the injector system 124. In another embodiment, the injector system 124 is controlled by a user interface 122 thereof to inject a prescribed amount of radioactivity into patients 132 who are scanned using a single scanner or multiple scanners.

In one embodiment, the processing unit 146 is operable to receive status information from, and send commands to the various components of the system 100 including the cyclotron 101, dispensing station 106, quality control device 110, injector systems 124, physiologic monitors 136, scanners 140, and computers 144. The processing unit 146 may also form part of the dose-calibrator 114.

Different types of stored data also may be communicated to the processing unit 146 as described herein. The data may be, for example, data relating to a prescribed dose for each patient 132 and the injection time for the patient 132. In still further embodiments, the data may include the type of radiopharmaceutical (e.g. oxygen-15), a predefined parametric equation, and/or a clinical protocol being followed in the medical procedure. The processing unit 146 may be configured to use the received data to calculate different values, for example, the SUV.

In one embodiment, the processing unit 146 may manage the process of producing the radiotracer 104 and delivering the radioisotope 102 according to the requirements of the imaging system 116. The processing unit 146 is capable of receiving information regarding an amount of a requested single dose 128, sending instructions to the cyclotron 101 to produce the individual quantity of the radioisotope 102 and/or sending instructions to the dispensing station to dispense the individual quantity of the radioisotope 102.

The processing unit 146 also may receive notification from the PM 136 of abnormalities in the measurements of the health of the patient 132, and consequently instructs the injector system 124, to halt infusion. In yet further embodiments, when the quality control unit 110 indicates that a quality is below acceptable minimum standards, the processing unit 146 provides notification to an operator and instructs the system 100 to purge the radiotracer from the injector system 124.

The processing unit 146 also may instruct the scanner 140 to initiate a scanning operation at an appropriate time after infusion by the injector system 124. The scanner 140 may follow a pre-defined set of acquisition procedures depending on a radiotracer and a clinical protocol being used. In one embodiment, the acquisition procedures may include initiation of scanning after a predefined time following injection of the radiotracer, introducing a pharmaceutical stress agent followed by injection of a radiotracer and imaging once again after a predefined time.

It should be noted that portions of the system 100 may be mounted inside a moveable structure with or without wheels in order to provide a portable or relocateable system. In one embodiment, a radiation shield 112 is mounted on a structure having wheels so the portions of the system within the radiation shield that are radioactive are more easily moved from one location to another.

Thus, the system 100 may be an integrated system for the production, quality control distribution and imaging using PET radiopharmaceuticals. The system 100 may centralize the management and control of the functions of preparing and injecting the radiotracer 104 into patient 132 and perform quantitative calculations based on radiopharmaceutical activity that is electronically and automatically stored and communicated. The system 100 further may provide an end-to-end control system and an integrated production, dispensing, quality control, infusion, data acquisition scheme in an automated manner.

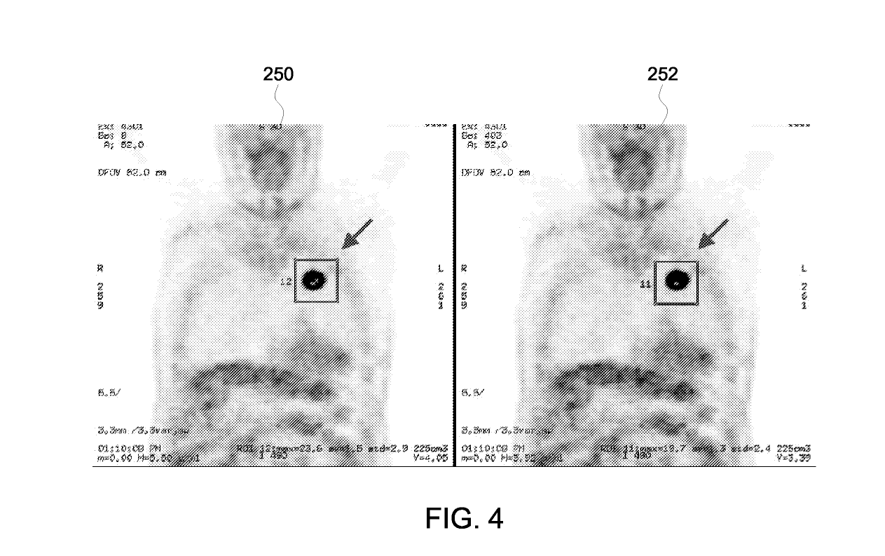

Using various embodiments, calibration errors may be reduced, for example, SUV image calibration errors. The reduction of calibration errors results in images that are more accurate and provide increased clinically relevant information. As an example, as shown in FIG. 4, the image 252 was reconstructed with less calibration errors than the image 250. As can been seen, a more clinically relevant image details may be shown in the image 252 than in the image 250.

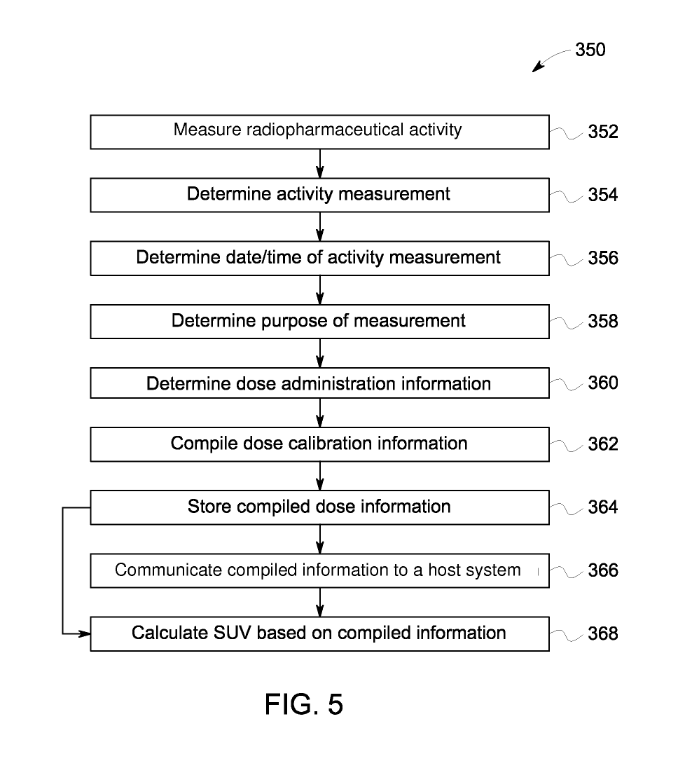

FIG. 5 is a method 350 for communicating dose calibration information, for example, from the dose calibrator 114 to the processing unit 146 or the host system 140 in accordance with one embodiment. The method 350 may communicate any type of radiopharmaceutical activity information or related information.

The method 350 begins by measuring radiopharmaceutical activity at 352 using a dose calibrator. At 354, the measured activity is determined using any suitable method. At 356, the date and/or time at which the radiopharmaceutical activity is determined. Next, at 358, a purpose or protocol for the measurement(s) may be determined. For example, the measurement may be performed before administration of the radiopharmaceutical to a subject, or may be made at the time of administration or after a medical procedure is performed (i.e., post-administration).

At 360, specific information relating to the dose administration may be determined. For example, the information may include patient information and the type of radionuclide/radiopharmaceutical injected into the patient. The user interface 122 may be used to input patient information and radiopharmaceutical information. For example, the patient information may include a patient's name, sex, age, weight and the like.

At 362, the information determined and at 352-360 is automatically compiled into a determined format. For example the information may be compiled into a DICOM file format. For example, the dose calibration information may be encoded as described in more detail herein.

At 364, the compiled information is stored. For example, the information may be stored in the storage device 118 which may be within the dose calibrator 114. The compiled dose information is transferred to a host system using a communication network at 366.

It should be noted that different types of information may be communicated in connection with the dose calibration information. For example, data or quality check information also may be communicated. In various embodiments, the communications protocol may be configured to provide a check that the correct dose calibrator measurement is being made. For example, data check information may be communicated regarding of check of whether if the dose calibrator is set for a particular radiopharmaceutical (e.g., fluorine-18), if that is the radiopharmaceutical required for the scanner protocol. Thus, the data check information may be used to determine whether the correct measurement information is communicated based on an expected type of radiopharmaceutical that should have been administered. However, any type of data integrity/quality checks may be provided, for example, clock settings (e.g., correct time synchronization between devices), or gross errors in the dose information, among other information. For example, the various embodiments may provide clock synchronizations such as ensuring that the correct reference time is used for the scanner time and the time used to indicate when the dose measurement was made. Thus, a synchronization of check of the synchronization between a scanner time and dose calibrator time (or clock associated with the dose calibrator) may be provided. As another example, and with respect to gross errors in dose information, the data check information may be used to confirm or check that an administration activity is what is expected (e.g., within a margin), namely that the correct data was transferred. Thus, if the data communicated exceeds a threshold or is not within a range based on a standard for a specific protocol, a warning may be provided, which may be sent when the data is communicated.

It also should be noted that at 368, the SUV may be calculated based on the compiled information stored on a storage device. It also should be noted that the information determined at steps 352-360 in various embodiments also may be automatically stored as described herein before compiling.

At least one technical effect of various embodiments is increased accuracy and/or reduced errors in the communication of dose calibration information.

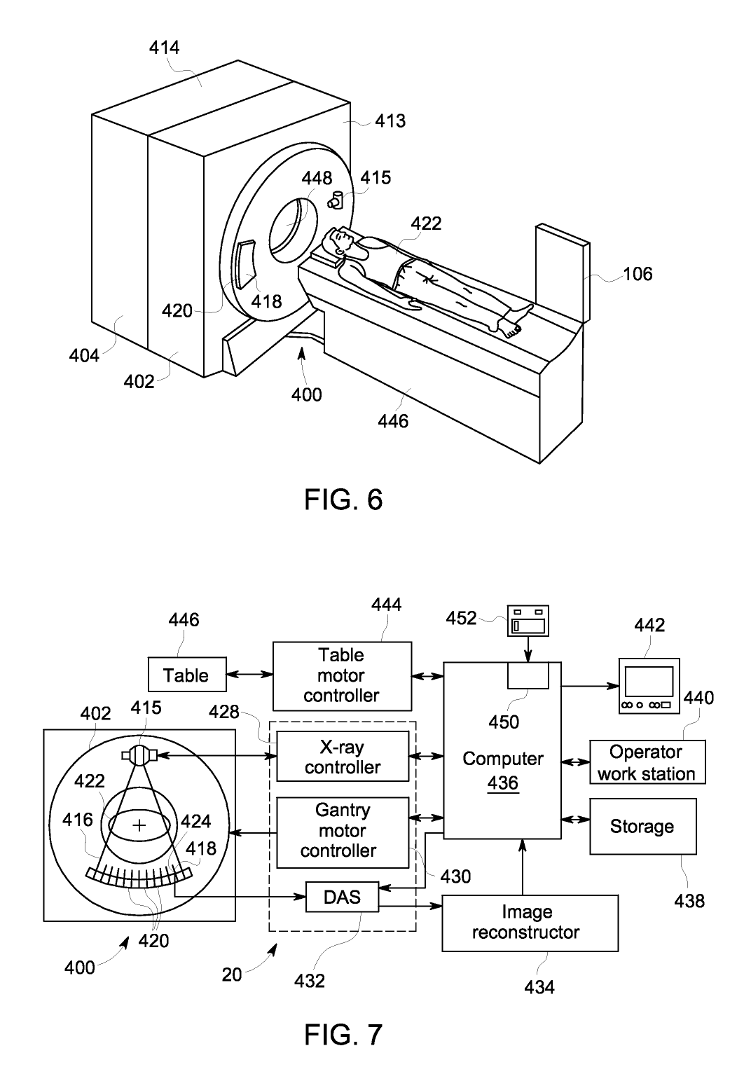

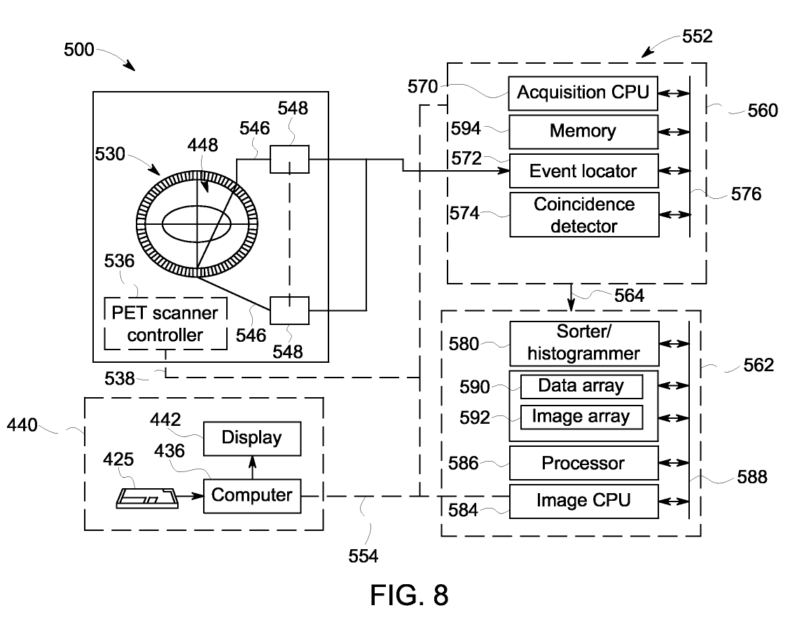

FIG. 6 is a perspective view of an exemplary imaging system 400 in accordance with an embodiment. FIG. 7 is a schematic block diagram of a portion of the imaging system 400 (shown in FIG. 6) and FIG. 8 is a schematic block diagram of another portion of the imaging system 400. In particular, in the exemplary embodiment, the imaging system 400 is a multi-modality or multi-modal imaging system and includes a first modality unit 402 and a second modality unit 404. The modality units 402 and 404 enable system 400 to scan an object, for example, the subject 422 (e.g., patient), in a first modality using the first modality unit 402 and to scan the subject 422 in a second modality using the second modality unit 404. The system 400 allows for multiple scans in different modalities to facilitate an increased diagnostic capability over single modality systems. The subject 422 also may be connected to the dispensing station 106 and information communicated using the system 100 as described in more detail herein.

In one embodiment, the multi-modal imaging system 400 is a CT/PET imaging system 400. The CT/PET system 400 includes a first gantry 413 associated with the first modality unit 402 and a second gantry 414 associated with the second modality unit 404. In other embodiments, modalities other than CT and PET may be employed with imaging system 400. The gantry 413 includes the first modality unit 402 that has an x-ray source 415 that projects a beam of x-rays 416 toward a plurality of detector elements 420 on the opposite side of the gantry 413.

In one embodiment, and referring specifically to the CT imaging modality portion shown in FIG. 7, the multi-modal imaging system 400 comprises a plurality of collimators 418 positioned between the subject 422 and the plurality of detector elements 420, wherein the collimators 418 having a tapered configuration as described herein. The tapered collimators 418 may be used to collimate x-ray radiation from x-ray tube. In an alternate embodiment, the collimators 418 may comprise x-ray absorbing material. The collimators 418 are assembled so that the adjacent collimators 418 form channels 424 therein for restricting background radiation from reaching the detectors.

The detector elements 420 may be formed by a plurality of detector rows (not shown) that together sense the projected x-rays that pass through an object, such as the subject 422. Each detector element 420 produces an electrical signal that represents the intensity of an impinging x-ray beam and therefore, allows estimation of the attenuation of the beam as the beam passes through the subject 422.

During a scan, to acquire x-ray projection data, the gantry 413 and the components mounted thereon rotate about an examination axis 426. FIG. 7 shows only a single row of detector elements 420 (i.e., a detector row). However, a detector array may be configured as a multislice detector array having a plurality of parallel rows of detector elements 420 such that projection data corresponding to a plurality of slices can be acquired simultaneously during a scan.

The rotation of the gantry 413, and the operation of x-ray source 415, are controlled by the system controller 423 of the CT/PET system 400. The system controller 423 includes an x-ray controller 428 that provides power and timing signals to the x-ray source 415 and a gantry motor controller 430 that controls the rotational speed and position of the gantry 413. A data acquisition system (DAS) 432 of the system controller 423 samples data from detector elements 420 for subsequent processing as described above. An image reconstructor 434 receives sampled and digitized x-ray projection data from the DAS 432 and performs high-speed image reconstruction. The reconstructed image is transmitted as an input to a computer 436 which stores the image in a storage device 438. The computer 436 may be programmed to implement various embodiments described herein. More specifically, the computer 436 may include an image reconstructor 434 that is programmed to carry out the various methods described herein.

The computer 436 also receives commands and scanning parameters from an operator via an operator workstation 440 that has an input device, such as, keyboard. The associated display 442 allows the operator to observe the reconstructed image and other data from the computer 436. The operator supplied commands and parameters are used by computer 436 to provide control signals and information to the DAS 432, the system controller 423, and the gantry motor controller 430. In addition, the computer 436 operates a table motor controller 444 which controls a motorized table 446 to position the subject 424 in the gantry 413 and 414. Specifically, the table 446 moves portions of the subject 422 through a gantry opening 448.

In one embodiment, the computer 436 includes a read/write device 450, for example, CD-ROM drive, DVD drive, magnetic optical disk (MOD) device, or any other digital device including a network connecting device such as an Ethernet device for reading instructions and/or data from a non-transitory computer-readable medium 452, such as a CD-ROM, a DVD or an other digital source such as a network or the Internet, as well as yet to be developed digital means. In another embodiment, the computer 436 executes instructions stored in firmware (not shown). The computer 436 is programmed to perform functions as described herein, and as used herein, the term computer is not limited to integrated circuits referred to in the art as computers, but broadly refers to computers, processors, microcontrollers, microcomputers, programmable logic controllers, application specific integrated circuits, and other programmable circuits, and these terms are used interchangeably herein. CT/PET system 400 also includes a plurality of PET detectors as described below including a plurality of detector elements.

FIG. 8 is a diagram of an exemplary PET imaging system 500 that may form one of the modalities of the multi-modality imaging system 400 described above. The PET imaging system 500 includes a detector ring assembly 530 including a plurality of detector scintillators. The detector ring assembly 530 includes the central opening 410, in which an object or patient, such as the subject 422 may be positioned, using, for example, the motorized table 446 (not shown in FIG. 6). The scanning operation is controlled from the operator workstation 440 through a PET scanner controller 536. A communication link 538 may be hardwired between the PET scanner controller 536 and the workstation 440. Optionally, the communication link 538 may be a wireless communication link that enables information to be transmitted to or from the workstation 440 to the PET scanner controller 536 wirelessly. In the exemplary embodiment, the workstation 440 controls real-time operation of the PET imaging system 500. The workstation 440 is also programmed to perform medical image diagnostic acquisition and reconstruction processes described herein. The operator workstation 440 may include the central processing unit (CPU) or computer 436, the display 442 and an input device 425. As used herein, the term “computer” may include any processor-based or microprocessor-based system configured to execute the methods described herein.

The methods described herein may be implemented as a set of instructions that include various commands that instruct the computer 436 as a processing machine to perform specific operations such as the methods and processes of the various embodiments described herein.

During operation of the exemplary detector 530, when a photon collides with a scintillator on the detector ring assembly 530, the absorption of the photon within the detector produces scintillation photons within the scintillator. The scintillator produces an analog signal that is transmitted on a communication link 546 when a scintillation event occurs. A set of acquisition circuits 548 is provided to receive these analog signals. The acquisition circuits 548 produce digital signals indicating the 3-dimensional (3D) location and total energy of each event. The acquisition circuits 548 also produce an event detection pulse, which indicates the time or moment the scintillation event occurred.

The digital signals are transmitted through a communication link, for example, a cable, to a data acquisition controller 552 that communicates with the workstation 440 and the PET scanner controller 536 via a communication link 554. In one embodiment, the data acquisition controller 552 includes a data acquisition processor 560 and an image reconstruction processor 562 that are interconnected via a communication link 564. During operation, the acquisition circuits 548 transmit the digital signals to the data acquisition processor 560. The data acquisition processor 560 then performs various image enhancing techniques on the digital signals and transmits the enhanced or corrected digital signals to the image reconstruction processor 562 as discussed in more detail below.

In the exemplary embodiment, the data acquisition processor 560 includes at least an acquisition CPU or computer 570. The data acquisition processor 560 also includes an event locator circuit 572 and a coincidence detector 574. The acquisition CPU 570 controls communications on a back-plane bus 576 and on the communication link 564. During operation, the data acquisition processor 560 periodically samples the digital signals produced by the acquisition circuits 548. The digital signals produced by the acquisition circuits 548 are transmitted to the event locator circuit 572. The event locator circuit 572 processes the information to identify each valid event and provide a set of digital numbers or values indicative of the identified event. For example, this information indicates when the event took place and the position of the scintillator that detected the event. The events are also counted to form a record of the single channel events recorded by each detector element. An event data packet is communicated to the coincidence detector 574 through the back-plane bus 576.

The coincidence detector 574 receives the event data packets from the event locator circuit 572 and determines if any two of the detected events are in coincidence. Coincident event pairs are located and recorded as a coincidence data packets by the coincidence detector 574. The output from the coincidence detector 574 is referred to herein as image data. In one embodiment, the image data may be stored in a memory device that is located in the data acquisition processor 560. Optionally, the image data may be stored in the workstation 440.

The image data subset is then transmitted to a sorter/histogrammer 580 to generate a data structure known as a histogram. The image reconstruction processor 562 also includes a memory module 582, an image CPU 584, an array processor 586, and a communication bus 588. During operation, the sorter/histogrammer 580 performs the motion related histogramming described above to generate the events listed in the image data into 3D data. This 3D data, or sinograms, is organized in one exemplary embodiment as a data array 590. The data array 590 is stored in the memory module 582. The communication bus 588 is linked to the communication link 576 through the image CPU 584. The image CPU 584 controls communication through communication bus 588. The array processor 586 is also connected to the communication bus 588. The array processor 586 receives the data array 590 as an input and reconstructs images in the form of image arrays 592. Resulting image arrays 592 are then stored in the memory module 582. The images stored in the image array 592 are communicated by the image CPU 584 to the operator workstation 440. In the illustrated embodiment, the PET imaging system 500 also includes a memory 594 that may be utilized to store a set of instructions to implement the various methods described herein.

The various embodiments and/or components, for example, the modules, or components and controllers therein, such as of the imaging system 400, may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, Reduced Instruction Set Computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program, which may form part of a tangible non-transitory computer readable medium or media. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, the embodiments are by no means limiting and are exemplary embodiments. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for communicating dose calibration information, the method comprising:
determining dose calibration information of a radiopharmaceutical at a dose calibrator;
automatically storing the dose calibration information in a storage device; and
communicating the stored dose calibration information to a host system.

2. The method of claim 1, wherein determining the dose calibration information comprises at least one of measuring dose calibration information pre-patient injection or measuring dose calibration information post-patient injection.

3. The method of claim 1, wherein the host system is at least one of an image acquisition system, an image reconstruction system, or an information system.

4. The method of claim 1, further comprising receiving a user input of patient information; and
linking the patient information to a measured radioactivity and a time of measurement.

5. The method of claim 1, further comprising calculating a standard update value (SUV) using the stored dose calibration information.

6. The method of claim 5, wherein the SUV is calculated at the dose calibrator.

7. The method of claim 1, further comprising configuring the dose calibrator to administer the radiopharmaceutical to a patient.

8. The method of claim 1, further comprising communicating the stored dose calibration information in at least one of a Digital Image Communication in Medicine (DICOM), flat file or printable barcode format.

9. The method of claim 1, further comprising communicating the stored dose calibration information using one of a Universal Serial Bus (USB), a Recommended Standard 232 (RS-232) interface, an Ethernet or a cloud computing network.

10. The method of claim 1, wherein the host system is remote from the dose calibrator.

11. The method of claim 1, wherein the dose calibrator is integrated with the host system.

12. The method of claim 1, further comprising automatically extracting the dose calibration information and storing the dose calibration information in the storage device, wherein the storage device is one of within the dose calibrator or remote from the dose calibrator.

13. The method of claim 1, further comprising communicating data check information with the dose calibration information to the host system.

14. A system for communicating radiopharmaceutical activity information, the system comprising:
a dose calibrator to determine dose calibration information of a radiopharmaceutical;
a storage device for storing the dose calibration information, wherein the dose calibrator is configured to automatically store the dose calibration information within the storage device; and
a host system communicatively coupled to the dose calibrator.

15. The system of claim 14, wherein the host system is at least one of an image acquisition system, an image reconstruction system, or an information system.

16. The system of claim 14, further comprising a user interface to input patient information that is lined to a measured radioactivity and a time of measurement based on a user input.

17. The system of claim 14, further comprising a processing unit configured to calculate a standard update value (SUV).

18. The method of claim 17, wherein the processing unit is part of the dose calibrator.

19. The system of claim 18, wherein the dose calibrator is configured to administer the radiopharmaceutical to a patient.

20. The system of claim 14, wherein the dose calibration information is stored in at least one of a DICOM, flat file, or printable barcode format.

21. The system of claim 14, wherein the dose calibrator is configured to communicate the stored dose calibration information to the host system using one of a Universal Serial Bus (USB), a Recommended Standard 232 (RS-232) interface, an Ethernet or a cloud computing network.

22. The system of claim 14, wherein the host system is located remote from the dose calibrator.

23. The system of claim 14, wherein the storage device is one of within the dose calibrator or remote from the dose calibrator.

24. The system of claim 14, wherein the storage device further stores data check information in connection with the dose calibration information for communication to the host system.
